(19) United States
(12) Patent Application Publication
(10) Pub. No.: US 2009/0312439 A1
(43) Pub. Date: Dec. 17, 2009

(54) USE OF ROLL COMPACTED PYROGENICALLY PRODUCED SILICON DIOXIDE IN PHARMACEUTICAL COMPOSITIONS

(76) Inventors: Ralph Hofmann, Buchen (DE); Ann Gray, Hanau (DE); Margarete Drechsler, Gelnhausen (DE)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/299,984

(22) PCT Filed: May 10, 2006
(86) PCT No.: PCT/EP2006/062216

§ 371 Date: Feb. 27, 2009

(51) Int. Cl. A61K 47/04

(52) U.S. Cl. 514/770

(57) ABSTRACT

The use of Schülpen based on pyrogenically produced silicon dioxide in pharmaceutical compositions, the pharmaceutical compositions per se, as well as an adsorbate consisting of the Schülpen and at least one further substance selected from pharmaceutical active constituents and excipients, and the produce of such adsorbates, are described.

### INTRODUCTION AND BACKGROUND

The present invention relates to the use of Schülpen of pyrogenic silicic acid in pharmaceutical compositions. The Schülpen are used in this connection in particular as carriers of pharmaceutical active constituents and/or excipients.

Pharmaceutical formulations contain in addition to the actual active constituent a number of further constituents, the so-called auxiliary substances or excipients, in order to convert the active constituent into suitable preparations that are effective at the desired point of use. A problem with many medicaments is their low solubility in water, resulting in a poor bioavailability and thereby often in an inadequate efficacy. In order to increase their solubility they may be adsorbed on suitable matrices having a high surface area. Pyrogenic silicic acids for example are suitable for this purpose, and are characterised by a high purity and inert behaviour compared to other active constituents and excipients. They also adsorb numerous active drug compounds reversibly. Pyrogenic silicic acids correspond to the pharmacopoeia monographs for highly dispersed silicon dioxide (for example European Pharmocopoeia Monograph No. 437) and may be used without any restrictions in pharmaceutical products.

It is known that for example by applying ethinyl oestradiol to pyrogenic silicic acid, its release rate can be significantly improved (product leaflet “Pigments” No. 19, Degussa AG). For example, the sorbate of 5.2 mg of this active constituent an 100 mg of pyrogenic silicic acid (AEROSIL 200, Degussa AG) an contact with water releases so much active constituent that a supersaturated solution is formed. An equivalent amount of the pure active constituent reaches the saturation equilibrium value of 1.1 mg/100 ml only after shaking over several days.

Numerous further AEROSIL 200 sorbates exhibit an improved active constituent release behaviour, for example those of griseofulvin (H. Rupprecht, M. J. Biersack, G. Kindl, Koll.-Z Z. Polym. 252 (1974) 415), indomethacin, aspirin, sulfaethidole, reserpine, chloramphenicol, oxolinic acid, probucol and hydrochlorothiazide (D. C. Monkhouse, J. L. Lach, J. Pharm. Sci., 57 (1968) 2143). Also, digitoxin-silicic acid matrices are characterised by an increased bioavailability compared to the pure active constituent (H. Flasch, B. Asmussen, N. Heinz, Arzneim. Forschung/Drug. Res. 28 (1978) 326).

In addition to the improvement in the bioavailability of sparingly soluble medicaments, carrier materials such as pyrogenic silicic acid may also be used in order to protect active constituents against environmental influences such as for example atmospheric oxygen, light or moisture and thereby stabilise them. For example, A. Y. Gore et al. in J. Pharm. Sci. 68 (1979) 197 describe the stabilisation of acetylsalicylic acid against hydrolysis by means of highly dispersed silicic acid. A targeted or delayed release of active constituent may also be achieved by adsorption onto a carrier.

Watanabe T et al. relates to the formulation of solid compositions comprising indomethacin and silica. The indomethacin is physically mixed with the silica by co-grinding or melting resulting in amorphous form of indometacin (Watanabe T et al. Stability of amorphous indomethacin compounded with silica, Int J Pharm, 226, 2001, pages 81-91).

Watanabe T et al. also relates to the formulation of solid compositions comprising indomethacin and silica. The indomethacin is physically mixed with the silica by co-grinding or melting resulting in amorphous form of indomethacin (Watanabe T et al. Prediction of apparent equilibrium solubility of indometacin compounded with silica by 13C solid state NMR′, Int J Pharm, 248, 2002, pages 123-129).

Watanabe T et al relates to the formulation of solid compositions comprising indomethacin and silica or PVP. The indomethacin is physically mixed with the silica or PVP by co-grinding or melting resulting in amorphous form of indometacin (Watanabe T et al. Comparison between Poly-vinylpyrrolidone and silica nano particles as carriers for indomethacin in a solid state dispersion. Int J Pharm, 250, 2003, pages 283-286).

GB 1 365 661 relates to the formulation of solid compositions comprising a drug substance with low water solubility (Cholesteryl beta-glucoside) and a carrier (Silica AEROSIL®). The composition is prepared by dissolving the betaglucoside in hot ethanol, subsequently adding this solution to the carrier powder and then evaporating the solvent from the resulting slurry. The resulting composition has a slower release rate than conventional formulations.

Takeuchi et al. relates to the formulation of compositions wherein the drug compound (tolbutamide) is present in amorphous form (Takeuchi et al. A spherical solid dispersion containing amorphous tolbutamide embedded in enteric coating polymers or colloidal silica was prepared by a spray-drying technique. Chem Pharm Bulletin Pharm Soc Japan. 35, 1987, pages 3800-3806).

Chowdary K et al. relates to the formulation of solid dispersions (powders) prepared by dissolving the drug (Meloxicam) in a solvent in the presence of carrier (Silica, AEROSIL). The solvent is then evaporated to dryness. The process of evaporating the solvent to dryness will result in the drug precipitating onto the carrier (Chowdary K et al. Enhanced of dissolution reate of meloxicam. Indian J. Pharm Sci, 63, 2001, page 105-154).

Nakakami H relates to the formulation of solid dispersions comprising poorly water-soluble drugs and nonporous fumed silicon dioxide as the carrier (Nakakami H. Solid dispersions of indomethacin and griseofulvin in nonporous fumed silicon dioxide, prepared by melting. Chem Pharm Bulletin Pharm Sei Japan, 39, 1991, pages 24172421).

Monkhouse D C et al. relates to the formulation of fine powders of a drug and a carrier (fumed silica). The drug and the silica are mechanically mixed under addition of an organic volatile solvent (acetone, chloroform or methylene chloride) in order to totally dissolve the drug in the Sample. The solvent is then evaporated to dryness. As the solvent is evaporated to dryness the drug will precipitate onto the carrier (Monkhouse D C et al. Use of adsorbents in enhancement of drug dissolution 1. J Pharm Sci, Am Pharm Ass Washington, 61, 1972, 1430-1435).

U.S. Pat. No. 6,217,909 describes an excipient composition comprising a particulate agglomerate of coprocessed microcrystalline cellulose and silicon dioxide, which can be a fumed silica powder.

U.S. Pat. No. 5,879,706 describes a tablet comprising at least 50% by weight valaciclovir and 0.05 to 3% by weight colloidal silicon dioxide, which can be a fumed silica.

US 2005/0207990 describes a powdery composition, which comprises lipophilic drugs such as steroided molecules and amorphous silica, having a specific surface area of at least 250 m²/g, wherein the steroridal molecules is moleculary dispersed in a solvent.

The amorphous silica can be Aeroperl®, which is an amorphous granulated silica with a silicon dioxide content of over 99.8% by weight.

WO 03/037379 (=US 2004/022844) describes the use of pyrogenically materials based on pyrogenically produced silicon dioxide in pharmaceutical compositions.

The granular material can be produced by dispersing the pyrogenically produced silicon dioxide in water and then spray drying the dispersion. The thus produced granular material exhibits the following physic-chemical datas:
Pore volume: 0.5 to 2.5 ml/g
Pore size distribution: less than 0.5% of the overall pore volume has a pore diameter of less than 5 nm, the remainder being mesopores and macropores.
pH value: 3.6 to 8.5
Tamped density: 220 to 700 g/l

US 2005/0096390 describes pharmaceutical compositions in particulate form or in solid dosage forms using a sorption material, which is normally a particulate material in the form of powders, particles, granules granulates etc.

Such particulate material has a bulk density of about 0.15 g/cm³ or more.

Furthermore this material has an oil absorption value of at least about 100 g Oil/100 g such as.

This particulate can be a silicon dioxide product that has the properties corresponding to Aeroperl® 300.

However, the use of pyrogenic silicic acids employed hitherto in pharmaceutical preparations does have some disadvantages. For example, a considerable amount of dust is formed during processing, which necessitates a complicated and expensive handling procedure. Furthermore available pyrogenic silicic acid has a low bulk density and tamped density and is therefore bulky to transport and store. Also, available adsorbates of pyrogenic silicic acid and a medicament often have an insufficient flowability and an unknown active constituent release behaviour on account of a very broad grain size distribution dependent an their processing.

Other types of carrier silica, such as precipitated silica, may not be used in many pharmaceuticals since they do not conform to pharmacopoeial requirements for purity and moisture content. The presence of increased amounts of moisture and impurities in precipitated silica can result in undesired reaction with actives and/or hydrolysis of moisture-sensitive active drug compounds.

### SUMMARY OF THE INVENTION

The object of the present invention is accordingly to provide an excipient for use in pharmaceutical compositions that does not exhibit the aforementioned disadvantages and also satisfies the stringent requirements of the pharmaceutical industry as regards purity and product safety.

This object is achieved by the use of a Schülpen based on pyrogenically produced silicon dioxide in a pharmaceutical composition. The present invention also provides a pharmaceutical composition that contains Schülpen based on pyrogenically produced silicon dioxide and at least one pharmaceutical active constituent. In addition, the present invention is directed to an adsorbate consisting of Schülpen based an pyrogenically produced silicon dioxide and at least one further substance selected from pharmaceutical active constituents and excipients, and to the production of such adsorbates.

The subject of the invention is the use of Schülpen based on pyrogenically produced silicon dioxide in a pharmaceutical composition.

In one embodiment of the invention, the pyrogenically produced silicon dioxide which has been compacted to Schülpen can have a tamped density (according to DIN EN ISO 787-11) of from 185 to 700 g/l.

In a preferred embodiment of the invention, the tamped density (according to DIN EN ISO 787-11) can be 200 to 450 g/l.

The more or less band-like intermediate products which are formed by pressing of the starting material during roll compactions are called Schülpen. They are comminuted in a second step.

The properties of the Schülpen can be influenced by the procedural parameters, such as the process control mode provided, the compacting force, the width of the nip between the two rolls and the pressure holding time established by the corresponding change in the speeds of rotation of the pressing rolls.

Compacting is understood as meaning mechanical compression without the addition of binders. In a particular embodiment of the invention, the Schülpen have a defined shape, it being possible for the size distribution to be adjusted by means of sieving.

The pyrogenically produced silicon dioxide which has been compacted to Schülpen and is employed according to the invention has a high transportation stability.

The pyrogenically produced silicon dioxide which has been compacted to Schülpen and has a tamped density (according to DIN EN ISO 787-11) of from 185 to 700 g/l can be prepared by pre-deaerating, or pre-compressing, pyrogenically produced silicon dioxide, compacting it to Schülpen, breaking up the Schülpen and optionally classifying the resulting material.

### BRIEF DESCRIPTION OF DRAWINGS

A schematic diagram of the process is shown in FIG. 1.

FIG. 2 illustrates a device for carrying out compacting according to the invention;

FIG. 3 illustrates a device for determination of dust content according to the invention;

FIG. 4 is a graph of fine dust content of Shülpen produced in accordance with the invention;

FIG. 5 is a graph of particle size distribution; and

FIG. 6 is a photomicrograph of the Schülpen of the invention.

Detailed Description of Invention According to FIG. 1, in the “pre-deaeration” step the pyrogenically produced silicon dioxide is deaerated or pre-compressed by means of known methods and devices. This step is necessary if a non-compressed pyrogenically produced, optionally freshly prepared silicon dioxide is employed.

If an already pre-compressed pyrogenically produced silicon dioxide is employed, this step of pre-deaeration can be omitted.

The pre-deaerated pyrogenically produced silicon dioxide is compressed (compacted) to the desired tamped density in the “compacting” step.

After the compacting, the Schülpen are broken up. The resulting material can then optionally be classified or sieved.

The fine content obtained during the sieving can be recycled back into the pre-deaeration step.

Either a non-compressed or a pre-compressed silicon dioxide can be employed as the educt in the pre-deaeration.

The pre-deaeration can be carried out either before the transportation or during the transportation to the compacting.

Before the transportation to the compacting, the pre-deaeration can be carried out by means of a vacuum-charged tube of a sinter material, such as, for example, sinter metal.

The pre-deaeration can furthermore be carried out in the transporting screw, it being possible for the transporting screw to be downstream of the device comprising a vacuum-charged tube.

In a further embodiment, the transporting screw can be employed as the sole device for the pre-deaeration.

The pre-deaeration can furthermore be carried out by means of a transporting screw arranged within a tube which is charged with vacuum. The vacuum-charged tube can comprise a sinter jacket, such as, for example, sinter metal.

If the device comprises a pre-deaeration tube, for example of a vacuum-charged tube, and a transporting screw arranged downstream, the pre-deaeration can be carried out in the tube if a non-compressed silicon dioxide is employed.

If a pre-compressed silicon dioxide is employed, the pre-deaeration can likewise be carried out in the tube. This pre-deaeration step can also be omitted.

If exclusively the transporting screw is employed for the pre-deaeration, pre-compressed silicon dioxide must be employed.

If the device which has a transporting screw within a vacuum-charged tube is employed for the pre-deaeration, both the non-compressed silicon dioxide and the pre-compressed silicon dioxide can be employed.

The pre-deaeration of the pyrogenically produced silicon dioxide can furthermore be carried out by means of filtration on a filter medium, such as, for example, a cloth or sinter material, such as, for example, sinter metal, sinter plastic, sinter ceramic or porous glass, with continuous removal of the filter cake by, for example, a conveying screw or a scraper. In one embodiment of the invention, a sinter metal tube with a metering screw can be used.

The pre-deaeration can furthermore be carried out by means of sedimentation, the breaking up of solid bridges being assisted by superimposed vibration, sound or slow stirring.

A hydrophilic pyrogenically produced silicon dioxide or a hydrophobic pyrogenically produced silicon dioxide can be employed as the educt.

The hydrophobic pyrogenically produced silicon dioxide can be prepared by means of surface modification.

The surface modification can be carried out with one or more compounds from the following group:
a) Organosilanes of the type (RO)₃Si(CₙH₂ₙ₊₁) and (RO)₃Si (CₙH₂ₙ₋₁)
   R=alkyl, such as, for example, methyl-, ethyl-, n-propyl-, i-propyl-, butyl-
   n=1-20
b) Organosilanes of the type R′ₓ(RO)_{y}si(CₙH₂ₙ₊₁) and R′ₓ(RO)_{y}Si(CₙH₂ₙ₋₁)
   R=alkyl, such as, for example, methyl-, ethyl-, n-propyl-, i-propyl-, butyl-
   R′=alkyl, such as, for example, methyl-, ethyl-, n-propyl-, i-propyl-, butyl-
   R′=cycloalkyl
   n=1-20
   x+y=3
   x=1,2
   y=1,2
c) Halogeno-organosilanes of the type X₃Si(CₙH₂ₙ₊₁) and X₃Si (CₙH₂ₙ₋₁)
   X=Cl, Br
   n=1-20
d) Halogeno-organosilanes of the type X₂(R′)Si(CₙH₂ₙ₊₁) and X₂(R′)Si(CₙH₂ₙ₋₁)
   X=Cl, Br
   R′=alkyl, such as, for example, methyl-, ethyl-, n-propyl-, i-propyl-, butyl-
   R′=cycloalkyl
   n=1-20
e) Halogeno-organosilanes of the type X(R′)₂Si(CₙH₂ₙ₊₁) and X(R′)₂Si(CₙH₂ₙ₋₁)
   X=Cl, Br
   R′=alkyl, such as, for example, methyl-, ethyl-, n-propyl-, i-propyl-, butyl-
   R′=cycloalkyl
   n=1-20
f) Organosilanes of the type (RO)₃Si(CH₂)ₘ—R′
   R=alkyl, such as methyl-, ethyl-, propyl-
   m=0,1-20
   R′=methyl-, aryl (for example —C₆H₅, substituted phenyl radicals)
      —C₄F₉, OCF₂—CHF—CF₃, —C₆F₁₃, —O—CF₂—CHF₂
      —NH₂, —N₃, —SCN, —CH═CH₂, —NH—CH₂—CH₂—NH₂,
      —N—(CH₂—CH₂—NH₂)₂
      —OOC(CH₃)C═CH₂
      —OCH₂—CH(O)CH₂
      —NH—CO—N—CO—(CH₂)₅
      —NH—COO—CH₃, —NH—COO—CH₂—CH₃, —NH—(CH₂)₃Si(OR)₃
      —Sₓ—(CH₂)₃Si(OR)₃, wherein x can be 1 to 10 and R can be alkyl, such as methyl-, ethyl-, propyl-, butyl-
      —SH
      —NR′R″R′″(R′=alkyl, aryl; R″=H, alkyl, aryl; R′″=H, alkyl, aryl, benzyl, C₂H₄NR″″R′″″ where R″″=H, alkyl and R′″″=H, alkyl)
g) Organosilanes of the type (R″)ₓ(RO)_{y}Si(CH₂)ₘ—R′

$$\begin{matrix}{R}^{″} = alkyl \\ = cycloalkyl\end{matrix}$$

$$x + y = 2$$

$$x = 1 , 2$$

$$y = 1 , 2$$

$$m = 0 , 1 to 20$$

R′=methyl-, aryl (for example —C₆H₅ , substituted phenyl radicals)
   —C₄F₉, —OCF₂—CHF—CF₃, —C₆F₁₃, —O—CF₂—CHF₂
   —NH₂, —N₃, —SCN, —CH═CH₂, —NH—CH₂—CH₂—NH₂,
   —N—(CH₂—CH₂—NH₂)₂
   —OOC(CH₃)C═CH₂
   —OCH₂—CH(O)CH₂
   —NH—CO—N—CO—(CH₂)₅
   —NH—COO—CH₃, —NH—COO—CH₂—CH₃, —NH—(CH₂)₃Si(OR)₃
   —Sₓ—(CH₂)₃Si(OR)₃, wherein x can be 1 to 10 and R can be methyl-, ethyl-, propyl-, butyl-
   —SH—NR′R″R′″ (R′=alkyl, aryl; R″=H, alkyl, aryl; R′″=H, alkyl, aryl, benzyl, C₂H₄NR″″R′″″ where R″″=H, alkyl and R′″″=H, alkyl)
h) Halogeno-organosilanes of the type X₃Si(CH₂)ₘ—R′
   X=Cl, Br
   m=0,1-20
   R′=methyl-, aryl (for example —C₆H₅, substituted phenyl radicals)
      —C₄F₉, —OCF₂—CHF—CF₃, —C₆F₁₃, —O—CF2—CHF₂
      —NH₂, —N₃, —SCN, —CH═CH₂,
      —NH—CH₂—CH₂—NH₂
      —N—(CH₂—CH₂—NH₂)₂
      —OOC(CH₃)C═CH₂
      —OCH₂—CH(O)CH₂
      —NH—CO—N—CO—(CH₂)₅
      —NH—COO—CH₃, —NH—COO—CH₂—CH₃, —NH—(CH₂)₃Si(OR)₃
      —Sₓ—(CH₂)₃Si(OR)₃, wherein x can be 1 to 10 and R can be methyl-, ethyl-, propyl-, butyl-
      —SH
i) Halogeno-organosilanes of the type (R)X₂Si(CH₂)ₘ—R′
   X=Cl, Br
   R=alkyl, such as methyl,- ethyl-, propyl-
   m=0,1-20
   R′=methyl-, aryl (e.g. —C₆H₅, substituted phenyl radicals)
      —C₄F₉, —OCF₂—CHF—CF₃, —C₆F₁₃, —O—CF₂—CHF₂
      —NH₂, —N₃, —SCN, —CH═CH₂, —NH—CH₂—CH₂—NH₂,
      —N—(CH₂—CH₂—NH₂)₂
      —OOC(CH₃)C═CH₂
      —OCH₂—CH(O)CH₂
      —NH—CO—N—CO—(CH₂)₅
      —NH—COO—CH₃, —NH—COO—CH₂—CH₃, —NH—(CH₂)₃Si(OR)₃, wherein R can be methyl-, ethyl-, propyl-, butyl-
      —Sₓ—(CH₂)₃Si(OR)₃, wherein R can be methyl-, ethyl-, propyl-, butyl- and x can be 1 to 10
      —SH
j) Halogeno-organosilanes of the type (R)₂XSi(CH₂)ₘ—R′
   X=Cl, Br
   R=alkyl, such as methyl,- ethyl-, propyl-, butyl-
   m=0,1-20
   R′=methyl-, aryl (e.g. —C₆H₅, substituted phenyl radicals)
      —C₄F₉, —OCF₂—CHF—CF₃, —C₆F₁₃, —O—CF₂—CHF₂
      —NH₂, —N₃, —SCN, —CH═CH₂, —NH—CH₂—CH₂—NH₂,
      —N—(CH₂—CH₂—NH₂)₂
      —OOC(CH₃)C═CH₂
      —OCH₂—CH(O)CH₂
      —NH—CO—N—CO—(CH₂)₅
      —NH—COO—CH₃, —NH—COO—CH₂—CH₃, —NH—(CH₂)₃Si(OR)₃
      —Sₓ—(CH₂)₃Si(OR)₃, wherein x can be 1 to 10 and R can be methyl-, ethyl-, propyl-, butyl-
      —SH
k) Silazanes of the type

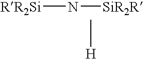

R=alkyl
R′=alkyl, vinyl
l) Cyclic polysiloxanes of the type D 3, D 4, D 5, wherein D 3, D 4 and D 5 are understood as cyclic polysiloxanes with 3, 4 or 5 units of the type —O—Si(CH₃)₂—.
   E.g. octamethylcyclotetrasiloxane=D 4

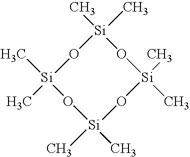

m) Polysiloxanes or silicone oils of the type

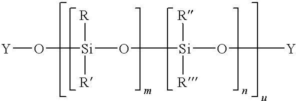

m=0,1,2,3, . . . ∞
n=0,1,2,3, . . . ∞
u=0,1,2,3, . . . ∞
Y=CH₃, H, CₙH₂ₙ₊₁ n=1-20
Y=Si(CH₃)₃, Si(CH₃)₂H Si(CH₃)₂OH, Si (CH₃)₂(OCH₃) Si(CH₃)₂(CₙH₂ₙ₊₁) n=1-20
R=alkyl, such as CₙH₂ₙ₊₁, wherein n=1 to 20, aryl, such as
   phenyl and substituted phenyl radicals, (CH₂)ₙ—NH₂, H
R′=alkyl, such as CₙH₂ₙ₊₁, wherein n=1 to 20, aryl, such as
   phenyl- and substituted phenyl radicals, (CH₂)ₙ—NH₂, H
R″=alkyl, such as CₙH₂ₙ₊₁, wherein n=1 to 20, aryl, such as
   phenyl- and substituted phenyl radicals, (CH₂)ₙ—NH₂, H
R′″=alkyl, such as CₙH₂ₙ₊₁, wherein n=1 to 20, aryl, such as
   phenyl und substituted phenyl radicals, (CH₂)ₙ—NH₂, H

In one embodiment, a pre-compressed pyrogenically produced silicon dioxide can be employed as the educt.

The non-compressed pyrogenically produced silicon dioxide employed can have a tamped density (according to DIN EN ISO 787-11) of less than 50 g/l, preferably of from 20 to 30 g/l. The pre-compressed pyrogenically produced silicon dioxide employed can have a tamped density (according to DIN EN ISO 787-11) of from 50 to 190 g/l, preferably 100 to 150 g/l, it being possible for the tamped density (according to DIN EN ISO 787-11 of a pre-compressed hydrophobic pyrogenically produced silicon dioxide to be 90 to 120 g/l.

The hydrophilic silicon dioxide employed can have a tamped density (according to DIN EN ISO 787-11) in the non-compressed state of less than 50 g/l, preferably 20 to 30 g/l.

In the pre-compressed state, the hydrophilic silicon dioxide can have a tamped density (according to DIN EN ISO 787-11) of from 50 to 190 g/l, preferably of from 100 to 150 g/l.

In the pre-compressed state, the hydrophobic silicon dioxide can have a tamped density (according to DIN EN ISO 787-11) of from 50 to 190 g/l, preferably of from 90 to 120 g/l.

The pyrogenically produced silicon dioxide employed can have a primary particle size of from 5 to 50 nm and a BET surface area of from 40 to 400 m²/g, preferably 100 to 250 m²/g.

The water content of the pyrogenically produced silicon dioxide employed can be less than 1 wt. %.

The pyrogenically produced silicon dioxide can be pre-compressed by means of known processes and devices. Thus, for example, the devices according to U.S. Pat. No. 4,325,686, U.S. Pat. No. 4,877,595, U.S. Pat. No. 3,838,785, U.S. Pat. No. 3,742,566, U.S. Pat. No. 3,762,851, U.S. Pat. No. 3,860,682 can be used.

In one embodiment, a pyrogenically produced silicon dioxide which has been pre-compressed by means of a pressing band filter according to EP 0280851 B1 or U.S. Pat. No. 4,877,595 can be employed.

The pyrogenically produced silicon dioxide can be transported to the compacting, for example, by means of a screw.

This transportation represents forced guidance of the pyrogenically produced silicon dioxide into the roll nip of the compacting rolls. If no conveying screw is employed, a pre-compressed pyrogenically produced silicon dioxide must be employed.

If a conveying screw is used, the pyrogenically produced silicon dioxide can be non-precompressed, because a pre-deaeration takes place here.

To achieve high bulk densities of the Schülpen, a conveying screw and a pre-compressed pyrogenically produced silicon dioxide can be employed.

A screw of decreasing volume or of increasing pitch or of decreasing diameter can be employed as the conveying screw.

The conveying screw can be surrounded by a vacuum-charged tube. This tube can comprise a sinter jacket. The pre-deaeration of the silicon dioxide takes place here in the transporting screw simultaneously with the transportation into the roll nip.

The compacting to Schülpen can be carried out by means of two rolls, it being possible for one or also both of these simultaneously to have a deaeration function.

Preferably, two compacting rolls, which can be smooth, can be employed. They can also be profiled. The profile can be present either only on one compacting roll or on both compacting rolls.

The profile can comprise grooves parallel to the axis. Alternatively, it can comprise troughs (depressions) in any desired arrangement and of any desired construction.

In a further embodiment, at least one of the rolls can be a vacuum roll. In this embodiment, the roll can be covered with sinter metal.

In order to be able to achieve the deaeration function, the roll can be produced from sinter metal or covered with a filter medium, such as, for example, with a cloth.

If deaeration of the pyrogenically produced silicon dioxide by means of the rolls is possible, the additional pre-deaeration, which can take place in the conveying screw or the feed tube, can be omitted.

If the roll is used for the pre-deaeration, the roll can have a smooth or profiled surface, it being possible for this surface to be only slightly grooved in order to improve the product intake.

During the compacting, a uniform pressing of the pyrogenically produced silicon dioxide should be ensured, in order to obtain Schülpen of uniform density.

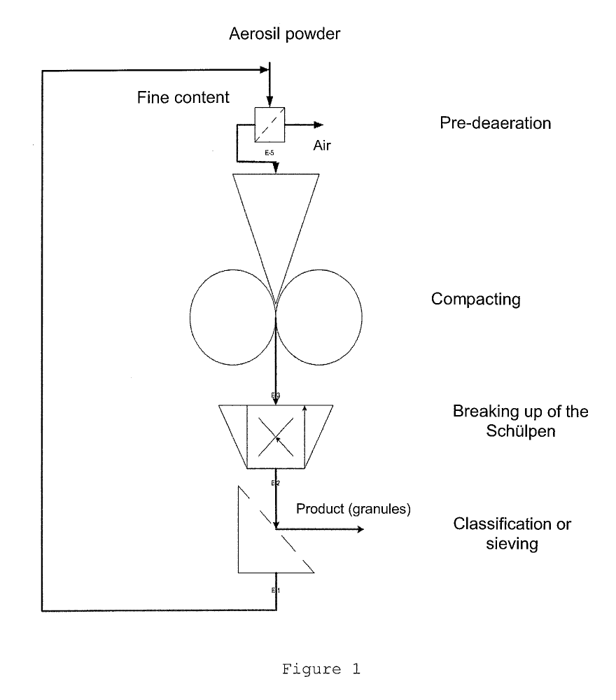

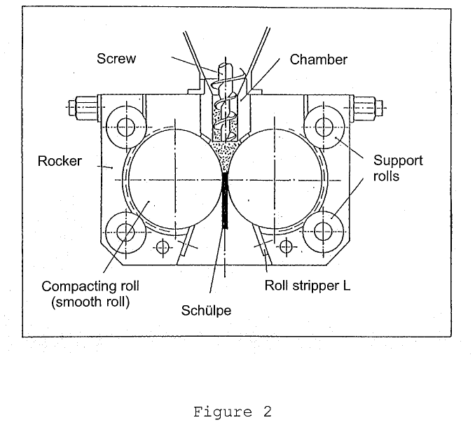

A device such as is shown in FIG. 2 can be employed for carrying out the compacting.

According to FIG. 2, the pyrogenically produced silicon dioxide is introduced by means of the pre-compressing screw 1 into the chamber 2 between the two rolls 3 and is pressed to Schülpen between the two rolls.

A device such as is described in the document DE AS 1807714 can furthermore be employed for carrying out the process.

Smooth rolls can preferably be employed in the compacting in order to avoid grit. It is furthermore possible to employ one or two rolls of sinter material, such as sinter metal or sinter ceramic, via which a deaeration can take place.

After the compacting, the Schülpen are broken up. A sieve granulator, which determines the particle size with its mesh width, can be used for this. The mesh width can be 250 μm to 20 mm.

A device with two rolls which rotate in opposite directions and have a defined nip or a toothed roll can furthermore be employed for breaking up the Schülpen.

The broken-up Schülpen can be classified by means of a sifter, a sieve or a classifier. The fine content (particles smaller than 200 μm) can be separated off by this procedure.

Transverse flow sifters, counter-flow deflection sifters etc. can be employed as the sifter.

A cyclone can be employed as the classifier.

The fine content (particles smaller than 200 μm) separated off during the classification can be recycled back into the process according to the invention.

Determination of the Dust Content

The dust content is determined in accordance with DIN 55992-2.

Before the measurement, a weighed amount of the Schülpen of the pyrogenically produced silicon dioxide to be analysed is introduced into a charging system at the top end of the down-tube. This is closed off downwards by a flap before the start of the measurement. The end of the down-tube is closed. With the start of the measurement, this flap is opened for a certain interval of time, so that the sample can fall into the down-pipe. While falling and on impinging on the base of the down-pipe, the sample releases dust into the air. The air turbulences during the fall ensure uniform distribution of the dust in the tube. Sedimentation of the suspended substances then starts. At the lower end of the down-tube, the light extinction caused by the suspended substance is measured by a photometric sensor. The course of the sedimentation is recorded as a function of the period of time by a PC. In the CIPACMT171 dust meter, the extinction E (in %) is plotted directly as a function of the duration of the sedimentation. In the SP3 dust meter from Lorenz, the dust index DI is calculated from the following formula (eq. 1) and is plotted as a function of time.

$${SZ}_{DI =} = 1 - \frac{{\int }_{1 s}^{30 s} E ( t ) d t - {\int }_{16 s}^{30 s} E ( t ) d t}{{\int }_{1 s}^{30 s} E ( t ) d t}$$

The dust index is a measure of the fine dust content of the sample. In this context, the fraction of which the sedimentation rate in air is less than 1 m/16 s=0.0625 m/s is called “fine dust”.

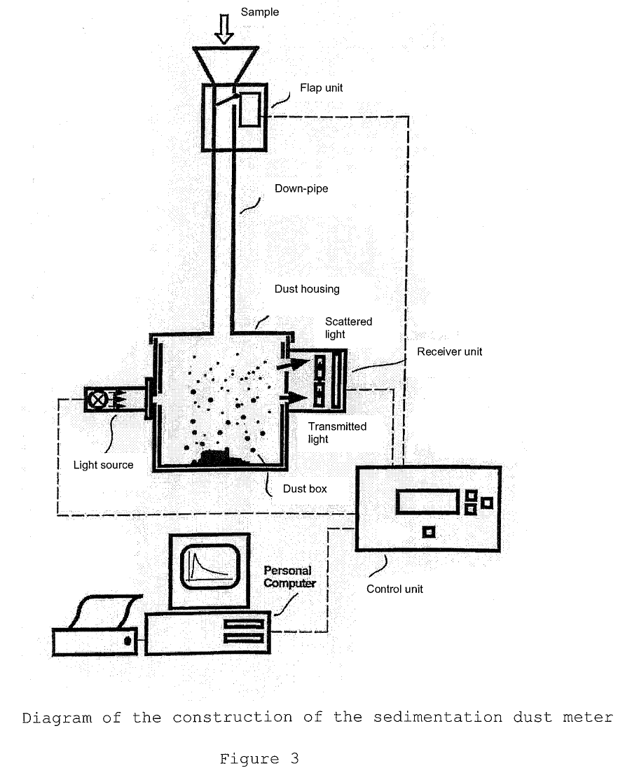

A schematic diagram of the device used for determination of the dust content is shown in FIG. 3.

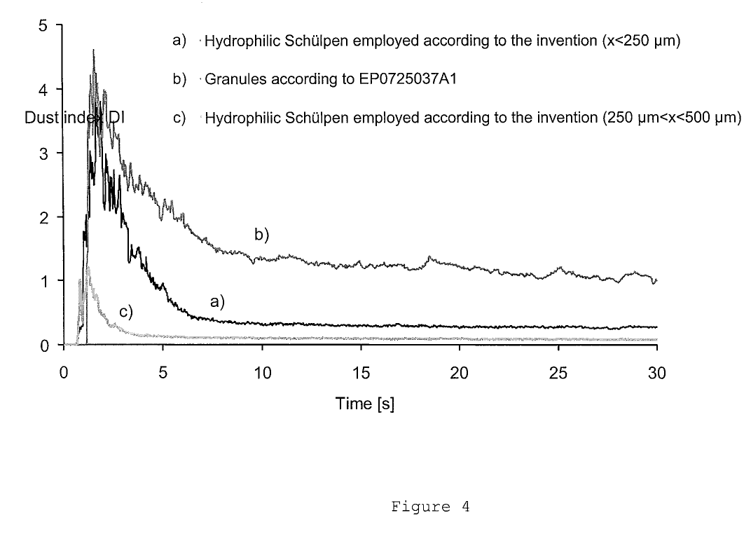

In FIG. 4, the fine dust content of the pyrogenically produced silicon dioxide which has been compacted to Schülpen and is employed according to the invention and the fine dust contents of pyrogenic silicon dioxide which has been compressed by another route are compared.

A pyrogenically produced silicon dioxide which was compressed by means of the pressing band filter according to EP 0280851 B1 was employed as the starting material for the silicon dioxide employed according to the invention.

FIG. 4 shows a measure of the particle size distribution and the average particle size of the bulk powder or Schülpen which are employed according to the invention with the. It is found here that the Schülpen of the pyrogenically produced silicon dioxide which are employed according to the invention sediment significantly better and form significantly less dust than the granules according to EP 0 725037 A1.

FIG. 4 furthermore shows a measure of the fine dust or suspended dust content. It is found here that the suspended dust content can be reduced drastically in the Schülpen employed according to the invention. In the case of granules according to EP 075 037 A1, a large content remains suspended for a very long time.

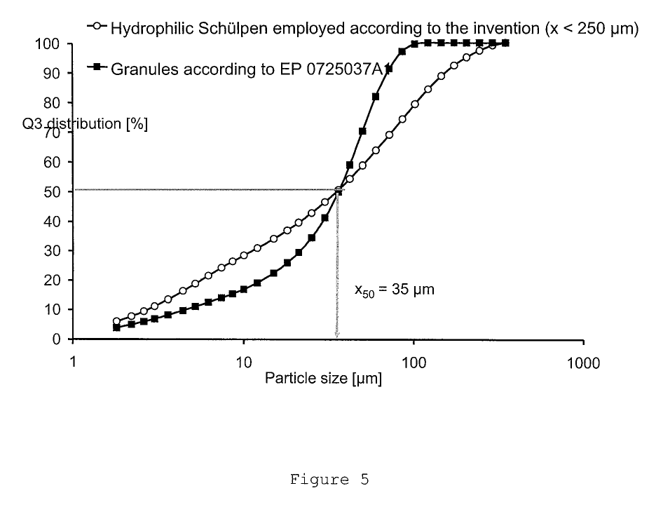

FIG. 5 shows the cumulative distribution (Q-3 distribution) of various granules according to EP 0 725 037 A1. None of these granules shows an average particle size of 120 μm. Only the largest particles in this group are between 96 and 128 μm in size. This is less than 11%.

The Schülpen employed according to the invention of X<250 μm have the same average particle size in laser diffraction spectroscopy as the granules according to EP 0 725 037 A1. In both cases this is ˜35 μm.

However, the Schülpen employed according to the invention generate significantly less dust.

The Schülpen fractions were produced by sieve granulation with a sieve of mesh width 500 μm and subsequent sieving on a 250 μm sieve. The fraction x<250 μm was the fine material in the sieving. The fraction having a particle size of between 250 and 500 μm was the coarse material.

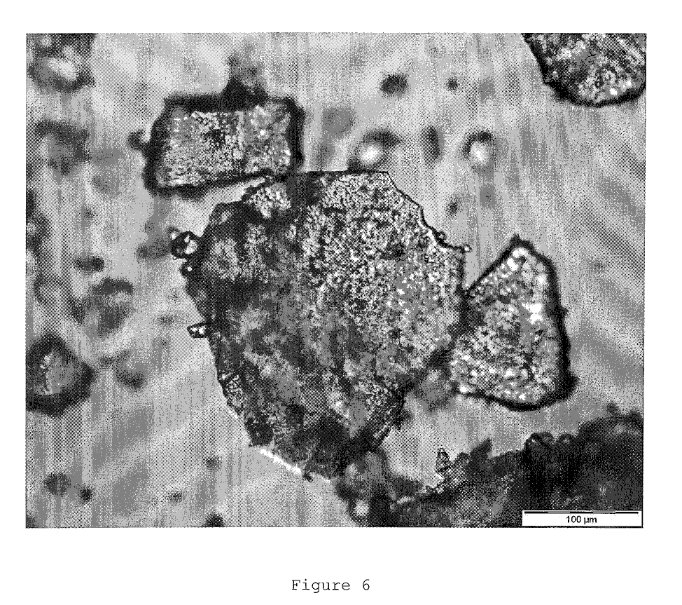

FIG. 6 shows the pyrogenically produced silicon dioxide which has been compacted to Schülpen and is employed according to the invention, in its granule form after the breaking up and sieving. It has an angular shape.

The granules according to DE 19601415 have a spherical appearance.

In a preferred embodiment, the Schülpen employed according to the invention have a tamped density of between 200 and 300 g/l. These Schülpen then have the necessary strength not to disintegrate again in the subsequent steps. However, they can readily be dispersed again.

The Schülpen obtained furthermore have a porosity.

After breaking up, the Schülpen employed according to the invention have an advantageous lack of dust even without sieving or classifying.

The Schülpen employed according to the invention have an agglomerate hardness of less than 50 N, measured with an ERWEKA 30.

The Schülpen employed according to the invention have no further dust content after being broken up. During handling, transportation or storage also, no further dust content forms in the Schülpen employed according to the invention. The pyrogenically produced silicon dioxide which has been compacted to Schülpen has no fine content with a diameter of less than 200 μm after the sieving.

The pyrogenically produced silicon dioxide which has been compacted to Schülpen has a lack of dust which is advantageous for all uses. It can be added to mixtures without loss and without dust pollution.

The pyrogenically produced silicon dioxide which has been compacted to Schülpen contains no binder.

The silicon dioxide utilized in the invention is of the very fine particle size variety. In the most preferred embodiments of the invention, the silicon dioxide utilized is a colloidal silicon dioxide. Colloidal silicon dioxide is a submicron fumed silica prepared by the vapor-phase hydrolysis (e.g., at 1110° C.) of a silicon compound, such as silicon tetrachloride. The product itself is a submicron, fluffy, light, loose, bluish-white, odorless and tasteless amorphous powder which is commercially available from a number of sources, including Cabot Corporation (under the tradename Cab-OSil); Degussa, Inc. (under the tradename AEROSIL); E.1. DuPont & Co.; and W.R. Grace & Co. Colloidal silicon dioxide is also known as colloidal silica, fumed silica, light anhydrous silicic acid, silicic anhydride, and silicon dioxide fumed, among others. A variety of commercial grades of colloidal silicon dioxide are produced by varying the manufacturing process. These modifications do not affect the silica content, specific gravity, refractive index, color or amorphous form. However, these modifications are known to change the particle size, surface areas, and bulk densities of the colloidal silicon dioxide products.

The surface area of the preferred class of silicon dioxides utilized in the invention ranges from about 50 m²/gm to about 500 m²/gm. The average primary particle diameter of the preferred class of silicon dioxides utilized in the invention ranges from about 5 nm to about 50 nm. However, in commercial colloidal silicon dioxide products, these particles are agglomerated or aggregated to varying extents. The bulk density of the preferred class of silicon dioxides utilized in the invention ranges from about 20 g/l to about 100 g/l.

Commercially available colloidal silicon dioxide products have, for example, a BET surface area ranging from about 50±15 m²/gm (AEROSIL OX50) to about 400±20 (Cab—O—Sil S-17) or 390±40 m²/gm (Cab—O—Sil EH-5). Commercially available particle sizes range from a nominal particle diameter of 7 nm (e.g., Cab—O—Sil S-17 or Cab—O—Sil EH-5) to an average primary particle size of 40 nm (AEROSIL OX50). The density of these products range from 72.0±8 g/l (Cab—O—Sil S-17) to 36.8 g/l (e.g., Cab—O—Sil M-5). The pH of these products at 4% aqueous dispersion ranges from pH 3.5-4.5.

The pyrogenic silicon dioxide serving as starting material is produced by feeding a volatile silicon compound through a nozzle into a detonating gas flame of hydrogen and air. Silicon tetrachloride is used in most cases. This substance hydrolyses under the influence of the water produced in the detonating gas reaction, to form silicon dioxide and hydrochloric acid. After leaving the flame the silicon dioxide enters a so-called coagulation zone in which the silicon dioxide primary particles and primary aggregates agglomerate. The product present as a form of aerosol in this stage is separated from the gaseous accompanying substances in cyclones and is then post-treated with moist hot air. The residual hydrochloric acid content can be reduced to below 0.025% by this process.

The granular materials (Schülpen) based an pyrogenically produced silicon dioxide may also be silanised. The carbon content of the granular material is then preferably 0.3 to 15.0 wt. %. Halogenated silantes, alkoxysilanes, silazanes and/or siloxanes may be used for the silanisation.

The following substances in particular may be used as halogenated silanes:
X=Cl, Br
n=1-20

halogenated organosilanes of the type X₂(R′)Si(CₙH₂ₙ₊₁) X=Cl, Br
R′=Alkyl
n=1-20

halogenated organosilanes of the type X(R′)₂Si(CₙH₂ₙ₊₁) X=Cl, Br
R′=Alkyl
n=1-20

halogenated organosilanes of the type X₃Si(CH₂)ₘ—R′
X=Cl, Br
m=0.1-20
R′=Alkyl, aryl (e.g. —C₆H₅)
   —C₄F₉, —OCF₂—CHF—CF₃, —C₆F₁₃, —O—CF₂—CHF₂
   —NH₂, —N₃, —SCN, —CH═CH₂,
   —OOC (CH₃)C═CH₂
   —OCH₂—CH(O)CH₂

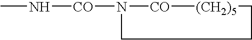

—NH—COO—CH₃, —NH—COO—CH₂—CH₃, —NH—(CH₂)₃Si(OR)₃
—Sₓ—(CH₂)₃Si(OR)₃
halogenated organosilanes of the type (R)X₂Si(CH₂)ₘ—R′
   X=Cl, Br
   R=Alkyl
   m=0.1-20
   R′=Alkyl, aryl (e.g. —C₆H₅)
      —C₄F₉, —OCF₂—CHF—CF₃, —C₆F₁₃, —O—CF₂—CHF₂
      —NH₂, —N₃, —SCN, —CH═CH₂,
      —OOC(CH₃)C═CH₂
      —OCH₂—CH(O)CH₂

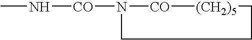

—NH—COO—CH₃, —NH—COO—CH₂—CH₃, —NH—(CH₂)₃Si(OR)₃
—Sₓ—(CH₂)₃Si(OR)₃
halogenated organosilanes of the type (R)₂XSi(CH₂)ₘ—R′
   X=Cl, Br
   R=Alkyl
   m=0.1-20
   R′=Alkyl, aryl (e.g. —C₆H₅)
      —C₄F₉, —OCF₂—CHF—CF₃, —C₆F₁₃, —O—CF₂—CHF₂
      —NH₂, —N₃, —SCN, —CH═CH₂,
      —OOC(CH₃)C═CH₂
      —OCH₂—CH(O)CH₂

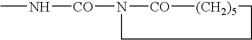

—NH—COO—CH₃, —NH—COO—CH₂—CH₃, —NH—(CH₂)₃Si(OR)₃
—Sₓ—(CH₂)₃Si(OR)₃

The following substances in particular may be used as alkoxysilanes:
organosilanes of the type (RO)₃Si(CₙH₂ₙ₊₁)
   R=Alkyl
   n=1-20
organosilanes of the type R′ₓ(RO)_{y}Si(CₙH₂ₙ₊₁)
   R=Alkyl
   R′=Alkyl
   n=1-20
   x+y=3
   x=1.2
   y=1.2
organosilanes of the type (RO)₃Si(CH₂)ₘ—R′
   R=Alkyl
   m=0.1-20
   R′=Alkyl, aryl (e.g. —C₆H₅)
      —C₄F₉, OCF₂—CHF—CF₃, —C₆F₁₃, —O—CF₂—CHF₂
      —NH₂, —N₃, —SCN, —CH═CH₂,
      —OOC(CH₃)C═CH₂
      —OCH₂—CH(O)CH₂

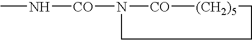

—NH—COO—CH₃, —NH—COO—CH₂—CH₃, —NH—(CH₂)₃Si(OR)₃
—Sₓ—(CH₂)₃Si(OR)₃
organosilanes of the type (R″)ₓ(RO)_{y}Si(CH₂)ₘ—R′
   R″=Alkyl
   x+y=2
   x=1.2
   y=1.2
   R=Alkyl, aryl (e.g. —C₆H₅)
      —C₄F₉, —OCF₂—CHF—CF₃, —C₆F₁₃, —O—CF₂—CHF₂
      —NH₂, —N₃, —SCN, —CH═CH₂,
      —OOC(CH₃)C═CH₂
      —OCH₂—CH(O)CH₂

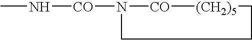

—NH—COO—CH₃, —NH—COO—CH₂—CH₃, —NH—(CH₂)₃Si(OR)₃
—Sₓ—(CH₂)₃Si(OR)₃

The silane Si 108 [(CH₃O)₃—Si—C₈H₁₇] trimethoxyoctylsilane may preferably be used as silanisation agent.

The following substances in particular may be used as silazanes:

Silazanes of the type:

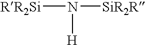

R=Alkyl
R′=Alkyl, vinyl

as well as for example hexamethyldisilazane.

The following substances in particular may be used as siloxanes:

cyclic polysiloxanes of the type D 3, D 4, D 5, e.g. octamethylcyclotetrasiloxane=D 4

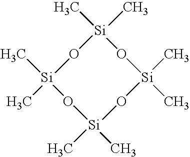

polysiloxanes and/or silicone oils of the type:

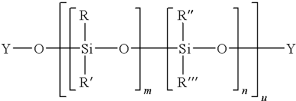

R=Alkyl, aryl, (CH₂)ₙ—NH₂, H
R′=Alkyl, aryl, (CH₂)ₙ—NH₂, H
R″=Alkyl, aryl, (CH₂)ₙ—NH₂, H
R′″=Alkyl, aryl, (CH₂)ₙ—NH₂, H
Y=CH₃, H, CₙH₂ₙ₊₁ where n=1-20
Y=Si(CH₃)₃, Si(CH₃)₂H
   Si(CH₃)₂OH, Si(CH₃)₂(OCH₃)
   Si(CH₃)₂(CₙH₂ₙ₊₁) where n=1-20
   m=0,1,2,3, . . . ∞
   n=0,1,2,3, . . . ∞
   u=0,1,2,3, . . . ∞

The silanisation may be carried out by spraying the Schülpen with the silanisation agent, which may optionally be dissolved in an organic solvent, for example ethanol, and then thermally treating the mixture at a temperature of 105° to 400° C. for a period of 1 to 6 hours.

An alternative method of silanising the Schülpen involves treating the Schülpen with the silanisation agent in vapour form and then thermally treating the mixture at a temperature of 200° to 800° C. for a period of 0.5 to 6 hours. The thermal treatment may be carried out under a protective gas, such as for example nitrogen.

The silanisation may be carried out continuously or batchwise in heatable mixers and dryers with spray devices. Suitable types of apparatus include for example ploughshare mixers, plate dryers, fluidised-bed dryers or turbulent-layer dryers.

The physicochemical parameters of the Schülpen, such as the specific surface, grain size distribution, pore volume, tamped density and silanol group concentration, pore distribution and pH value may be altered within the specified limits by varying the starting substances, spraying conditions, heat treatment and silanisation.

The Schülpen of pyrogenic silicon dioxide may be used according to the invention in any suitable solid, semi-solid or liquid dosage forms, preferably for oral and/or topical applications, for example in suspensions, emulsions, aerosols, ointments, creams, gels, pastes, suppositories, sticks, powders, topical powders, redispersible powders, powders used to make oral suspsensions, granules, tablets, pastilles, sugar-coated pills, film-coated tablets, filled hard gelatin capsules, soft gelatin capsules, extrudates, microcapsules or microspheres. Particularly preferred are solid dosage forms such as for example powders, granules, tablets and filled hard capsules. The expression “pharmaceutical composition” also covers within the scope of the present invention precursors and intermediates used for the production of granules, tablets, capsules, suspensions, dry ointments and dry drops. Such precursors and intermediates may for example also be in the form of a powder, granular material or extrudate.

Methods for the production of solid, semi-solid and liquid dosage forms are known and are described in numerous publications and textbooks relating to pharmaceutical technology, cf. for example K. H. Bauer, K. -H. Frömming, C. Fuhrer, Lehrbuch der pharmazeutischen Technologie, 6^{rh} Edition, Wissenschaftliche Verlagsgesellschaft mbH Stuttgart 1999.

The silicon dioxide Schülpen may be used in combination with any arbitrary pharmaceutical active constituent. The following may be mentioned by way of example:

α-proteinase inhibitor, abacavir, abciximab, acarbose, acetylsalicylic acid, acyclovir, adenosine, albuterol, aldesleukin, alendronate, alfuzosin, alosetrone, alprazolam, alteplase, ambroxol, amifostine, amiodarone, amisulprid, amlodipine, amoxicillin, amphetamine, amphotericin, ampicillin, amprenavir, anagrelide, anastrozole, ancrod, anti-haemophilia factor, aprotinin, atenolol, atorvastatin, atropine, azelastine, azithromycin, azulene, barnidipin, beclomethasone, benazepril, benserazide, beraprost, betamethasone, betaxolol, bezafibrate, bicalutamide, bisabolol, bisoprolol, botulinum toxin, brimonidine, bromazepam, bromocriptine, budesonide, bupivacaine, bupropion, buspirone, butorphanol, cabergoline, calcipotriene, calcitonin, calcitriol, camphor, candesartan, candesartan cilexetil, captopril, carbamazepine, carbidopa, carboplatin, carvedilol, cefaclor, cefadroxil, cefaxitin, cefazolin, cefdinir, cefepime, cefixime, cefmetazole, cefoperazone, cefotiam, cefoxopran, cefpodoxime, cefprozil, ceftazidime, ceftibuten, ceftriaxone, cefuroxime, celecoxib, celiprolol, cephalexin, cerivastatin, cetirizine, chloramphenicol, cilastatin, cilazapril, cimetidine, ciprofibrate, ciprofloxacin, cisapride, cisplatin, citalopram, clarithromycin, clavulanic acid, clindamycin, clomipramine, clonazepam, clonidine, clopidogrel, clotrimazole, clozapine, cromolyn, cyclophosphamide, cyclosporine, cyproterone, dalteparin, deferoxamine, desogestrel, dextroamphetamine, diazepam, diclofenac, didanosine, digitoxin, digoxin, dihydroergotamine, diltiazem, diphtheria protein, diphtheria toxoxide, divalproex, dobutamine, docetaxel, dolasetron, donepezil, dornase-a, dorzolamide, doxazosin, doxifluridin, doxorubicin, dydrogesterone, ecabet, efavirenz, enalapril, enoxaparin, eperisone, epinastin, epirubicin, eptifibatide, erythropoietin-a, erythropoietin-β, etanercept, ethinyl oestradiol, etodolac, etoposide, factor VIII, famciclovir, famotidine, faropeneme, felodipine, fenofibrate, fenoldopam, fentanyl, fexofenadin, filgrastim, finasteride, flomoxef, fluconazole, fludarabine, flunisolide, flunitrazepam, fluoxetine, flutamide, fluticasone, fluvastatin, fluvoxamine, follitropin-a, follitropin-β, formoterol, fosinopril, furosemide, gabapentin, gadodiamide, ganciclovir, gatifloxacin, gemcitabine, gestoden, glatiramer, glibenclamide, glimepiride, glipizide, glyburide, goserelin, granisetron, griseofulvin, hepatitis B antigen, hyaluronic acid, hycosin, hydrochlorothiazide, hydrocodone, hydrocortisone, hydromorphone, hydroxychloroquine, hylan G-F 20, ibuprofen, ifosfamide, imidapril, imiglucerase, imipenem, immunoglobulin, indinavir, indomethacin, infliximab, insulin, insulin human, insulin Lispro, insulin aspart, interferon β, interferon a, iodine 125, iodixanol, iohexol, iomeprol, iopromid, ioversol, ioxoprolen, ipratropium, ipriflavone, irbesartan, irinotecan, isosorbide, isotretinoin, isradipine, itraconazole, potassium chlorazepate, potassium chloride, ketorolac, ketotifen, whooping cough vaccine, coagulation factor IX, lamivudine, lamotrigine, lansoprazole, latanoprost, leflunomide, lenograstim, letrozole, leuprolide, levodopa, levofloxacin, levonorgestrel, levothyroxine, lidocaine, linezolid, lisinopril, lopamidol, loracarbef, loratadine, lorazepam, losartan, lovastatin, lysineacetylsalicylic acid, manidipin, mecobalamin, medroxyprogesterone, megestrol, meloxicam, menatetrenone, meningococcus vaccine, menotropine, meropenem, mesalamine, metaxalone, metformin, methylphenidate, methylprednisolone, metoprolol, midazolam, milrinone, minocycline, mirtazapine, misoprostol, mitoxantrone, moclobemid, modafinil, mometasone, montelukast, morniflumat, morphine, moxifloxacin, mycophenolate, nabumetone, nadroparin, naproxen, naratriptan, nefazodone, nelfinavir, nevirapine, niacin, nicardipine, nicergoline, nifedipine, nilutamide, nilvadipine, nimodipine, nitroglycerin, nizatidine, norethindrone, norfloxacin, octreotide, olanzapine, omeprazole, ondansetron, orlistate, oseltamivir, oestradiol, oestrogens, oxaliplatin, oxaprozin, oxolinic acid, oxybutynin, paclitaxel, palivizumab, pamidronate, pancrelipase, panipenem, pantoprazol, paracetamol, paroxetine, pentoxifylline, pergolide, phenytoin, pioglitazon, piperacillin, piroxicam, pramipexole, pravastatin, prazosin, probucol, progesterone, propafenone, propofol, propoxyphene, prostaglandin, quetiapine, quinapril, rabeprazol, raloxifene, ramipril, ranitidine, repaglinide, reserpine, ribavirin, riluzole, risperidone, ritonavir, rituximab, rivastigmin, rizatriptan, rofecoxib, ropinirol, rosiglitazone, salmeterol, saquinavir, sargramostim, serrapeptase, sertraline, sevelamer, sibutramin, sildenafil, simvastatin, somatropine, sotalol, spironolactone, stavudin, sulbactam, sulfaethidole, sulfamethoxazole, sulfasalazin, sulpirid, sumatriptan, tacrolimus, tamoxifen, tamsulosin, tazobactam, teicoplanin, temocapril, temozolomid, tenecteplase, tenoxicam, teprenon, terazosin, terbinafine, terbutaline, tetanus toxoid, tetrabenazine, tetrazepam, thymol, tiagabine, tibolon, ticarcillin, ticlopidine, timolol, tirofiban, tizanidine, tobramycin, tocopheryl nicotinate, tolterodine, topiramate, topotecan, torasemid, tramadol, trandolapril, trastuzumab, triamcinolone, triazolam, trimebutin, trimethoprim, troglitazone, tropisetrone, tulobuterol, unoproston, urofollitropine, valacyclovir, valproic acid, valsartan, vancomycin, venlafaxine, verapamil, verteporfin, vigabatrin, vinorelbine, vinpocetine, voglibose, warfarin, zafirlukast, zaleplon, zanamivir, zidovudine, zolmitriptan, zolpidem, zopiclone and their derivatives.

Pharmaceutical active constituents are however also understood to include other substances such as vitamins, provitamins, essential fatty acids, extracts of plant and animal origin and oils of plant and animal origin.

In the embodiments of the present invention, which include active ingredients, the active ingredients suitable for use in the pharmaceutical compositions and methods of the present invention are not particularly limited, as the compositions are surprisingly capable of effectively delivering a wide variety of active ingredients. The active ingredients can be hydrophilic, lipophilic, amphiphilic or hydrophobic, and can be solubilized, dispersed, or partially solubilized and dispersed, in the encapsulation coat. Alternatively, the active ingredient can be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, diagnostic agents, nutritional agents, and the like. It should be appreciated that the categorization of an active ingredient as hydrophilic or hydrophobic may change, depending upon the particular salts, isomers, analogs and derivatives used.

In one embodiment, the active ingredient agent is hydrophobic. Hydrophobic active ingredients are compounds with little or no water solubility. Intrinsic water solubilities (i.e., water solubility of the unionized form) for hydrophobic active ingredients are less than about 1% by weight, and typically less than about 0.1% or 0.01% by weight. In a particular aspect of this embodiment, the active ingredient is a hydrophobic drug. In other particular aspects, the active ingredient is a nutrient, a cosmeceutical, a diagnostic agent or a nutritional agent.

Suitable hydrophobic active ingredients are not limited by therapeutic category, and can be, for example, analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agent, anti-gout agents, anti-hypertensive agents, anti-malarials, anti-migraine agents, anti-muscarinic agents, anti-neoplastic agents, erectile dysfunction improvement agents, immunosuppresants, anti-protozoal agents, antithyroid agents, anxiolytic agents, sedatives, hypnotics, neuroleptics, β-blockers, cardiac inotropic agents, corticosteroids, diuretics, anti-parkinsonian agents, gastrointestinal agents, histamine receptor antagonists, keratolytics, lipid regulating agents, anti-anginal agents, Cox-2 inhibitors, leukotriene inhibitors, macrolides, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, extracts of plant or animal origin, oils of plant or animal origin, and mixtures thereof.

Specific, non-limiting examples of suitable hydrophobic active ingredients are: acetretin, albendazole, albuterol, aminoglutethimide, amiodarone, amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, efavirenz, eprosartan, ergocalciferol, ergotamine, essential fatty acid sources, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-thryroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nisoldipine, nilutanide, nitrofurantoin, nizatidine, omeprazole, oprevelkin, oestradiol, okaprozin, paclitaxel, pantoprazole, paracalcitol, paroxetine, pentazocine, pioglitazone, pizofetin, pravastatin, prednisolone, probucol, progesterone, pseudoephedrine, pyridostigmine, rabeprazole, raloxifene, repaglinide, rifabutine, rifapentine, rimexolone, ritanovir, rizatriptan, rofecoxib, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremifene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem, and zopiclone, nutritional oils, essential fatty acids, non-essential fatty acids, extracts of plant or animal origin, oils of plant or animal origin. Of course, salts, isomers and derivatives of the above-listed hydrophobic active ingredients may also be used, as well as mixtures thereof.

Among the above-listed hydrophobic active ingredients, preferred active ingredients include: acetretin, albendazole, albuterol, aminoglutethimide, amiodarone, amlodipine, amphetamine, amphotericin B, atorvastatin, atovaquone, azithromycin, baclofen, benzonatate, bicalutanide, busulfan, butenafine, calcifediol, calcipotriene, calcitriol, camptothecin, capsaicin, carbamezepine, carotenes, celecoxib, cerivastatin, chlorpheniramine, cholecaliferol, cimetidine, cinnarizine, ciprofloxacin, cisapride, cetirizine, clarithromycin, clemastine, clomiphene, codeine, coenzyme Q 10, cyclosporin, danazol, dantrolene, dexchlorpheniramine, diclofenac, digoxin, dehydroepiandrosterone, dihydroergotamine, dihydrotachysterol, dirithromycin, donezepil, efavirenz, ergocalciferol, ergotamine, esomeprazole, essential fatty acid sources, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, ibuprofen, irinotecan, isotretinoin, itraconazole, ivermectin, ketoconazole, ketorolac, lamotrigine, lansoprazole, leflunomide, loperamide, loratadine, lovastatin, L-thryroxine, lutein, lycopene, mifepristone, mefloquine, megestrol acetate, methdone, methoxsalen, metronidazole, miconazole, midazolam, miglitol, mitoxantrone, medroxyprogesterone, montelukast, nabumetone, nalbuphine, naratriptan, nelfinavir, nilutanide, nitrofurantoin, nizatidine, omeprazole, oestradiol, oxaprozin, paclitaxel, paracalcitol, pentazocine, pioglitazone, pizofetin, pravastatin, probucol, progesterone, pseudoephedrine, pyridostigmine, rabeprazole, raloxifene, rofecoxib, repaglinide, rifabutine, rifapentine, rimexolone, ritanovir, rizatriptan, rosiglitazone, saquinavir, sibutramine, sildenafil citrate, simvastatin, sirolimus, spironolactone, sumatriptan, tacrine, tacrolimus, tamoxifen, tamsulosin, targretin, tazarotene, teniposide, terbinafine, tetrahydrocannabinol, tiagabine, tizanidine, topiramate, topotecan, toremifene, tramadol, tretinoin, troglitazone, trovafloxacin, verteporfin, vigabatrin, vitamin A, vitamin D, vitamin E, vitamin K, zafirlukast, zileuton, zolmitriptan, zolpidem, zopiclone, pharmaceutically acceptable salts, isomers and derivatives thereof, and mixtures thereof as well as nutritional oils, essential fatty acids, non-essential fatty acids, extracts of plant or animal origin, oils of plant or animal origin.

Particularly preferred hydrophobic active ingredients include: acetretin, albuterol, aminoglutethimide, amiodarone, amlodipine, amprenavir, atorvastatin, atovaquone, baclofen, benzonatate, bicalutanide, busulfan, calcifediol, calcipotriene, calcitriol, camptothecin, capsaicin, carbamezepine, carotenes, celecoxib, chlorpheniramine, cholecaliferol, cimetidine, cinnarizine, cisapride, cetirizine, clemastine, coenzyme Q10, cyclosporin, danazol, dantrolene, dexchlorpheniramine, diclofenac, dehydroepiandrosterone, dihydroergotamine, dihydrotachysterol, efavirenz, ergocalciferol, ergotamine, essential fatty acid sources, etodolac, etoposide, famotidine, fenofibrate, fexofenadine, finasteride, fluconazole, flurbiprofen, fosphenytoin, frovatriptan, furazolidone, glibenclamide, glipizide, glyburide, glimepiride, ibuprofen, irinotecan, isotretinoin, itraconazole, ivermectin, ketoconazole, ketorolac, lamotrigine, lansoprazole, leflunomide, loperamide, loratadine, lovastatin, L-thryroxine, lutein, lycopene, medroxyprogesterone, mifepristone, megestrol acetate, methoxsalen, metronidazole, miconazole, miglitol, mitoxantrone, montelukast, nabumetone, naratriptan, nelfinavir, nilutanide, nitrofurantoin, nizatidine, omeprazole, oestradiol, oxaprozin, paclitaxel, paracalcitol, pioglitazone, pizofetin, pranlukast, probucol, progesterone, pseudoephedrine, rabeprazole, raloxifene, rofecoxib, repaglinide, rifabutine, rifapentine, rimexolone, ritanovir, rizatriptan, rosiglitazone, saquinavir, sildenafil citrate, simvastatin, sirolimus, tacrolimus, tamoxifen, tamsulosin, targretin, tazarotene, teniposide, terbenafine, tetrahydrocannabinol, tiagabine, tizanidine, topiramate, topotecan, toremifene, tramadol, tretinoin, troglitazone, trovafloxacin, ubidecarenone, vigabatrin, vitamin A, vitamin D, vitamin E, vitamin K, zafirlukast, zileuton, ziprasidone, zolmitriptan, pharmaceutically acceptable salts, isomers and derivatives thereof, and mixtures thereof.

Most preferred hydrophobic active ingredients include: amlodipine, amprenavir, atorvastatin, atovaquone, celecoxib, cisapride, coenzyme Q10, cyclosporin, famotidine, fenofibrate, fexofenadine, finasteride, ibuprofen, itraconazole, lansoprazole, loratadine, lovastatin, megestrol acetate, montelukast, nabumetone, nizatidine, omeprazole, oxaprozin, paclitaxel, paracalcitol, pioglitazone, pranlukast, progesterone, pseudoephedrine, rabeprazole, rapamycin, rofecoxib, repaglinide, rimexolone, ritanovir, rosiglitazone, saquinavir, sildenafil citrate, simvastatin, sirolimus, tacrolimus, tamsulosin, teniposide, terbenafine, tetrahydrocannabinol, tiagabine, tizanidine, tramadol, troglitazone, vitamin A, vitamin D, vitamin E, vitamin K, zafirlukast, zileuton, as well as nutritional oils, essential fatty acids, non-essential fatty acids, extracts of plant or animal origin, oils of plant or animal origin, pharmaceutically acceptable salts, isomers and derivatives thereof, and mixtures thereof.

In another embodiment, the active ingredient is hydrophilic. Amphiphilic compounds are also included within the glass of hydrophilic active ingredients. Apparent water solubilities for hydrophilic active ingredients are greater than about 0.1% by weight, and typically greater than about 1% by weight. In a particular aspect of this embodiment, the hydrophilic active ingredient is a hydrophilic drug. In other particular aspects, the hydrophilic active ingredient is a cosmeceutical, a diagnostic agent, or a nutritional agent.

Suitable hydrophilic active ingredients are not limited by therapeutic category, and can be, for example, analgesics, 30 anti-inflammatory agents, antihelminthics, anti-arrhythmic agents, anti-bacterial agents, anti-viral agents, anticoagulants, anti-depressants, anti-diabetics, anti-epileptics, anti-fungal agent, anti-gout agents, anti-hypertensive agents, anti-malarials, anti-migraine agents, anti-muscarinic agents, anti-neoplastic agents, erectile dysfunction improvement agents, immunosuppresants, anti-protozoal agents, antithyroid agents, anxiolytic agents, sedatives, hypnotics, neuroleptics, β-blockers, cardiac inotropic agents, corticosteroids, diuretics, anti-parkinsonian agents, gastro intestinal agents, histamine receptor antagonists, keratolytics, lipid regulating agents, anti-anginal agents, Cox-2 inhibitors, leukotriene inhibitors, macrolides, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof

Likewise, the hydrophilic active ingredients can be a cytokine, a peptidomimetic, a peptide, a protein, a toxoid, a serum, an antibody, a vaccine, a nucleoside, a nucleotide, a portion of genetic material, a nucleic acid, or a mixture thereof.

Specific, non-limiting examples of suitable hydrophilic active ingredients include: acarbose; acyclovir; acetyl cysteine; acetylcholine chloride; alatrofloxacin; alendronate; alglucerase; amantadine hydrochloride; ambenomium; amifostine; amiloride hydrochloride; aminocaproic acid; amphotericin B; antihemophilic factor (human); antihemophilic factor (porcine); antihemophilic factor (recombinant); aprotinin; asparaginase; atenolol; atracurium besylate; atropine; azithromycin; aztreonam; BCG vaccine; bacitracin; becalermin; belladona; bepridil hydrochloride; bleomycin Sulfate; calcitonin human; calcitonin salmon; carboplatin; capecitabine; capreomycin sulfate; cefamandole nafate; cefazolin sodium; cefepime hydrochloride; cefixime; cefonicid sodium; cefoperazone; cefotetan disodium; cefotaxime; cefoxitin sodium; ceftizoxime; ceftriaxone; cefuroxime axetil; cephalexin; cephapirin sodium; cholera vaccine; chorionic gonadotropin; cidofovir; cisplatin; cladribine; clidinium bromide; clindamycin and clindamyein derivatives; ciprofloxacin; clodronate; colistimethate sodium; colistin sulfate; corticotropin; cosyntropin; cromolyn sodium; cytarabine; dalteparin sodium; danaparoid; desferrioxamine; denileukin diftitox; desmopressin; diatrizoate meglumine and diatrizoate sodium; dicyclomine; didanosine; dirithromycin; dopamine hydrochloride; dornase alpha; doxacurium chloride; doxorubicin; etidronate disodium; enalaprilat; enkephalin; enoxaparin; enoxaparin sodium; ephedrine; epinephrine; epoetin alpha; erythromyein; esmolol hydrochloride; factor IX; famciclovir; fludarabine; fluoxetine; foscamet sodium; gamciclovir; granulocyte colony stimulating factor; granulocyte-macrophage stimulating factor; recombinant human growth hormones; bovine growth hormone; gentamycin; glucagon; glycopyrolate; gonadotropin releasing hormone and synthetic analogs thereof; GnRH; gonadorelin; grepafloxacin; haemophilus B conjugate vaccine; Hepatitis A virus vaccine inactivated; Hepatitis B virus vaccine inactivated; heparin sodium; indinavir sulfate; influenza virus vaccine; interleukin-2; interleukin-3; insulin-human; insulin lispro; insulin procine; insulin NPH; insulin aspart; insulin glargine; insulin detemir; interferon alpha; interferon beta; ipratropium bromide; ifosfamide; Japanese encephalitis virus vaccine; lamivudine; leucovorin calcium; leuprolide acetate; levofloxaein; lincomycin and lincomycin derivatives; lobucavir; lomefloxacin; loracarbef; mannitol; measles virus vaccine; meningococcal vaccine; menotropins; mepenzolate bromide; mesalamine; methenamine; methotrexate; methscopolamine; metformin hydrochloride; metoprolol; mezocilhn sodium; mivacurium chloride; mumps viral vaccine; nedocromil sodium; neostigmine bromide; neostigmine methyl sulfate; neurontin; norfloxacin; octreotide acetate; ofloxacin; olpadronate; oxytocin; pamidronate disodium; pancuronium bromide; paroxetine; perfloxacin; pentamidine isethionate; pentostatin; pentoxifylline; periciclovir; pentagastrin; phentolamine mesylate; phenylalanine; physostigmine salicylate; plague vaccine; piperacillin sodium; platelet derived growth factor; pneumococcal vaccine polyvalent; poliovirus vaccine (inactivated); poliovirus vaccine live (OPV); polymyxin B sulfate; pralidoxime chloride; pramlintide; pregabalin; propafenone; propantheline bromide; pyridostigmine bromide; rabies vaccine; residronate; ribavarin; rimantadine hydrochloride; rotavirus vaccine; salmeterol xinafoate; sincalide; small pox vaccine; solatol; somatostatin; sparfloxacin; spectinomycin; stavudine; streptokinase; streptozocin; suxamethonium chloride; tacrine hydrochloride; terbutaline Sulfate; thiopeta; ticarcillin; tiludronate; timolol; tissue type plasminogen activator; TNFR:Fc; TNK-tPA; trandolapril; trimetrexate gluconate; trospectinomycin; trovafloxacin; tubocurarine chloride; tumor necrosis factor; typhoid vaccine live; urea; urokinase; vancomycin; valacyclovir; valsartan; varicella virus vaccine live; vasopressin and vasopressin derivatives; vecuronium bromide; vinblastine; vincristine; vinorelbine; vitamin B12; warfarin sodium; yellow fever vaccine; zalcitabine; zanamivir; zolendronate; zidovudine; pharmaceutically acceptable salts, isomers and derivatives thereof; and mixtures thereof.

Among the above-listed hydrophilic active ingredients, preferred active ingredients include acarbose; acyclovir; atracurium besylate; alendronate; alglucerase; amantadine hydrochloride; amphotericin B; antihemophilic factor (human); antihemophilic factor (porcine); antihemophilic factor (recombinant); azithromycin; calcitonin human; calcitonin salmon; capecitabine; cefazolin sodium; cefonicid sodium; cefoperazone; cefoxitin sodium; ceftizoxime; ceftriaxone; cefuroxime axetil; cephalexin; chorionic gonadotropin; cidofovir; cladribine; clindamycin and clindamyein derivatives; corticotropin; cosyntropin; cromolyn sodium; cytarabine; dalteparin sodium; danaparoid; desmopressin; didanosine; dirithromycin; etidronate disodium; enoxaparin sodium; epoetin alpha; factor IX; famciclovir; fludarabine; foscarnet sodium; ganciclovir; granulocyte to colony stimulating factor; granulocyte-macrophage stimulating factor; recombinant human growth hormones; bovine growth hormone; gentamycin; glucagon; gonadotropin releasing hormone and synthetic analogs thereof; GnRH; gonadorelin; haemophilus B conjugate vaccine; Hepatitis A virus vaccine inactivated; Hepatitis B virus vaccine inactivated; heparin sodium; indinavir sulfate; influenza virus vaccine; interleukin-2; interleukin-3; insulin-human; insulin lispro; insulin procine; insulin NPH; insulin aspart; insulin glargine; insulin detemir; interferon alpha; interferon beta; ipratropium bromide; ifosfamide; lamivudine; leucovorin calcium; leuprolide acetate; lincomycin and lincomycin derivatives; metformin hydrochloride; nedocromil sodium; neostigmine bromide; neostigmine methyl sulfate; neurontin; octreotide acetate; olpadronate; pamidronate disodium; pancuronium bromide; pentamidine isethionate; pentagastrin; physostigmine salicylate; poliovirus vaccine live (OPV); pyridostigmine bromide; residronate; ribavarin; rimantadine hydrochloride; rotavirus vaccine; salmeterol xinafoate; somatostatin; spectinomycin; stavudine; streptokinase; ticarcillin; tiludronate; tissue type plasminogen activator; TNFR:Fc; TNK-tPA; trimetrexate gluconate; trospectinomycin; tumor necrosis factor; typhoid vaccine live; urokinase; vancomycin; valacyclovir; vasopressin and vasopressin derivatives; vinblastine; vincristine; vinorelbine; warfarin sodium; zalcitabine; zanamivir; zidovudine; pharmaceutically acceptable salts, isomers and derivatives thereof; and mixtures thereof.

Most preferred hydrophilic active ingredients include acarbose; alendronate; amantadine hydrochloride; azithro-40 mycin; calcitonin human; calcitonin salmon; ceftriaxone; cefuroxime axetil; chorionic gonadotropin; cromolyn sodium; dalteparin sodium; danaparoid; desmopressin; didanosine; etidronate disodium; enoxaparin sodium; epoetin alpha; factor IX; famciclovir; foscarnet sodium; ganciclovir; granulocyte colony stimulating factor; granulocytemacrophage stimulating factor; recombinant human growth hormones; bovine growth hormone; glucagon; gonadotropin releasing hormone and synthetic analogs thereof, GnRH; gonadorelin; heparin sodium; indinavir sulfate; influenza virus vaccine; interleukin-2; interleukin-3; insulin-human; insulin lispro; insulin procine interferon alpha; interferon beta; leuprolide acetate; metformin hydrochloride; nedocromil sodium; neostigmine bromide; neostigmine methyl Sulfate; neurontin; octreotide acetate; olpadronate; pamidronate disodium; residronate; rimantadine hydrochloride; salmeterol xinafoate; somatostatin; stavudine; ticarcillin; tiludronate; tissue type plasminogen activator; TNFR:Fc; TNKtPA; tumor necrosis factor; typhoid vaccine live; vancomycin; valacyclovir; vasopressin and vasopressin derivatives; zalcitabine; zanamivir; zidovudine; pharmaceutically acceptable salts, isomers and derivatives thereof; and mixtures thereof.

The active agent(s) which may be incorporated into solid dosage forms invention include systemically active therapeutic agents, locally active therapeutic agents, disinfecting agents, chemical impregnants, cleansing agents, deodorants, fragrances, dyes, animal repellents, insect repellents, a fertilizing agents, pesticides, herbicides, fungicides, and plant growth stimulants, and the like.

A wide variety of therapeutically active agents can be used in conjunction with the present invention. The therapeutically active agents (e.g. pharmaceutical agents) which may be used in the compositions of the present invention include both water soluble and water insoluble drugs. Examples of such therapeutically active agents include antihistamines (e.g., dimenhydrinate, diphenhydramine, chlorpheniramine and dexchlorpheniramine maleate), analgesics (e.g., aspirin, codeine, morphine, dihydromorphone, oxycodone, etc.), non-steroidal anti-inflammatory agents (e.g., naproxyn, diclofenac, indomethacin, ibuprofen, sulindac), antiemetics (e.g., metoclopramide), antiepileptics (e.g., phenytoin, meprobamate and nitrezepam), vasodilators (e.g., nifedipine, papaverine, diltiazem and nicardirine), anti-tussive agents and expectorants (e.g., codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluazide), antihypotensives (e.g., propranolol, clonidine), antihypertensives (e.g, clonidine, methyldopa), bronchodilators (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants, laxatives, vitamins, stimulants (including appetite suppressants such as phenylpropanolamine). The above list is not meant to be exclusive.

A wide variety of locally active agents can be used in conjunction with the novel excipient described herein, and include both water soluble and water insoluble agents. The locally active agent(s) which may be included in the controlled release formulation of the present invention is intended to exert its effect in the environment of use, e.g., the oral cavity, although in some instances the active agent may also have systemic activity via absorption into the blood via the surrounding mucosa.

The locally active agent(s) include antifungal agents (e.g., amphotericin B, clotrimazole, nystatin, ketoconazole, miconazol, etc.), antibiotic agents (Penicillins, cephalosporins erythromycin, tetracycline, aminoglycosides, etc.), antiviral agents (e.g, acyclovir, idoxuridine, etc.), breath fresheners (e.g. chlorophyll), antitussive agents (e.g., dextromethorphan hydrochloride), anti-cariogenic compounds (e.g., metallic salts of fluoride, sodium monofluorophosphate, stannous fluoride, amine fluorides), analgesic agents (e.g., methylsalicylate, salicylic acid, etc.), local anesthetics (e.g., benzocaine), oral antiseptics (e.g., chlorhexidine and salts thereof, hexylresorcinol, dequalinium chloride, cetylpyridinium chloride), anti-flammatory agents (e.g., dexamethasone, betamethasone, prednisone, prednisolone, triamcinolone, hydrocortisone, etc.), hormonal agents (oestriol), antiplaque agents (e.g, chlorhexidine and salts thereof, octenidine, and mixtures of thymol, menthol, methysalicylate, eucalyptol), acidity reducing agents (e.g., buffering agents such as potassium phosphate lo dibasic, calcium carbonate, sodium bicarbonate, sodium and potassium hydroxide, etc.), and tooth desensitizers (e.g., potassium nitrate). This list is not meant to be exclusive. The solid formulations of the invention may also include other locally active agents, such as flavorants and sweeteners. Generally any flavoring or food additive such as those described in Chemicals Used in Food Processing, pub 1274 by the National Academy of Sciences, pages 63-258 may be used.

A wide variety of pharmaceutically systemically active agents can be formulated e.g., vitamins, minerals, amino acids, essential trace elements, hormones and antagonists thereof, steroids, non-steroid anti-inflammatory agents, antineoplastic agents, antigens, antihistaminic agents, neuropharmacologic agents, including analgesics, vasodilators, anticoagulants, antimicrobial agents, antiviral agents, antifungal agents, antiparasitic agents, heavy metal antagonists, locally active drugs moderating the digestive tract, such as enzymes, antacids, histamine antagonists, diuretics and cardiovascular drugs.

The active drug substances can be as described in US 2005/0096390 A1. Accordingly, a first drug or active substance of the dosage forms and pharmaceutical compositions of this invention is fenofibrate as described above or an analog thereof. It should be understood that this invention includes dosage forms and compositions comprising a mixture of two, three or even four different fibrates and/or fibric acids. Examples of other useful fibrates are bezafibrate, ciprofibrate, clinofibrate, clofibrate, etofylline, clofibrate, fenofibrate, gemfibrozil, pirifibrate, simfibrate and tocofibrate; particularly useful are gemfibrozil, fenofibrate, bezafibrate, clofibrate, ciprofibrate and active metabolites and analogues thereof including any relevant fibric acid such as fenofibric acid.

A second drug or active substance of the dosage forms and pharmaceutical compositions of this invention can be pravastatin as described above or a pharmaceutically acceptable salt thereof such as the sodium salt.

A typical compound of the invention is characterised by being lipophilic and/or having a poor solubility in water at 25° C. In general, the compound has a solubility lower than 1 mg/ml in water at 25° C., such as lower than 0.5, 0.1, 0.05, or 0.01 mg/ml. Typically, the compound is an active pharmaceutical ingredient, such as a steroidal molecule and/or a hormone/anti-hormone in general. A large range of other active pharmaceutical ingredients may benefit from the present technology, such as albendazole, aminogluthethimide, aminosalicylic acids (3- 4- or 5-aminosalicylic acids) amiodarone, astemizole, azathioprine, beclamide, benorylate, benperidol, bezafibrate, biotin, bromocriptine, bromocriptine mesylate, bumetanide, busulphan, cabergoline, carbamazepine, cefixime, chenodeoxycholic acid, chlorambucil, chloroquine, chlorpropamide, chlorprothixene, chlorthalidone, cinnarizine, cinoxacin, clobazam, clofazimine, clofibrate, clonazepam, cyclopenthiazide, cyclosporin A, dapsone, demeclocycline, diazoxide, diflunisal, digitoxin, digoxin, disulfiram, domperidone, droperidol, enoxacin, epothilone, ethionamide, etretinate, felodipine, fenbufen, fexofenadine, flumazenil, folic acid, furosemide, glipizide, gliquidone, griseofulvin, haloperidol, hydrochlorothiazide, hydroflumethiazide, ibuprofen, iloprost, indomethacin, isocarboxazid, isosorbide dinitrate, isotretinoin, isradipine, itraconazole, ketazolam, ketoconazol, ketoprofen, lansoprazole, liothyronine sodium, lisuride, loperamide, loratadine, lorazepam, lovastatin, mebendazole, medazepam, mefenamic acid, menadione, mequitazine, methotrexate, misoprostol, morphine, niclosamide, nifedipine, nimodipine, nitrazepam, omeprazole, oxazepam, oxytetracycline, pantoprazole, perphenazine, phenylbutazone, pimozide, pindolol, probenecid, probucol, pyrantel embonate, pyrimethamine, retinol, riboflavin, simvastatin, stilboestrol, sulindac, sulphadiazine, sulphamethoxazole, sulphasalazine, sulpiride, tamoxifen, temazepam, thiabendazole, thioguanine, tocopherol, tolbutamide, tretinoin, triamteren, triazolam, trimethoprim and zopiclone.

As said, a compound of the invention is typically a steroidal molecule or otherwise a hormone of which can be mentioned:
androgens, such as testosterone and esters thereof (testosterone enanthate, testosterone undecanoate, testosterone cypionate, testosterone propionate)
estrogens/anti-estrogens, such as estradiol and esters thereof (estradiol valerate, estradiol enanthate, estradiol cypionate, estradiol undecylate), estriol, estrone, conjugated estrogens, equilin, ethinyl estradiol, fenestrel, mestranol, nylestriol, quinestrol, clomifene, estrogen receptor alpha agonists, estrogen receptor alpha antagonists, estrogen receptor beta agonists, estrogen receptor beta antagonists, estrogen receptor downregulators.
Corticosteroids, such as cortisones and glucocorticoids, e.g. beclomethasone dipropionate, betamethasone, betamethasone valerate, budesonide, clobetasol propionate, clobetasone butyrate, cortisone acetate, dexamethasone, fludrocortisone acetate, prednisolone, prednisone.
progestins/antiandrogens, such as cyproterone, drospirenone, etonogestrel, desogestrel, gestodene, levonorgestrel, norethisterones, norgestimate, norethindrone, norethindrone acetate, norethynodrel, norgestimate, norgestrel, medrogestone, medroxyprogesterone acetate, progesterone, progesterone receptor A specific ligands, progesterone receptor B specific ligands, mesoprogestins, antiprogestins, asoprisnil, asoprisnil ecamate.
Aldosterone antagonists, such as spironolactones, eplerenone, canrenoate, canrenone, dicirenone, mexrenoate, prorenoate, epostane, mespirenone, oxprenoate, spirorenone, spiroxasone, prorenone.
Vitamin D hormones, such as alfacalcidol, calcifediol, calciferol, calcitriol.

Thus, a steroidal molecule of the invention may be selected from estradiol and esters thereof, ethinyl estradiol, conjugated estrogens, testosterone and esters thereof, cyproterone, drospirenone, etonogestrel, desogestrel, gestodene, levonorgestrel, norethisterones, norgestimate, norethindrone, norethindrone acetate, norethynodrel, norgestimate, norgestrel, medrogestone, medroxyprogesterone acetate, progesterone, spironolactones, eplerenone, canrenoate, canrenone, dicirenone, mexrenoate, prorenoate, epostane, mespirenone, oxprenoate, spirorenone, spiroxasone, prorenone, asoprisnil, beclomethasone dipropionate, betamethasone, betamethasone valerate, budesonide, clobetasol propionate, clobetasone butyrate, cortisone acetate, dexamethasone, fludrocortisone acetate, prednisolone, prednisone, alfacalcidol, calcifediol, calciferol or calcitriol.

It is to be understood that the compositions of the invention may comprise more than one active drug substance, e.g. a combination of two ore more drug substances. For example, a composition of the invention may comprise a therapeutic effective dose of drospirenone and a therapeutic effective dose of an estrogen.

In currently interesting embodiments, the compound of the invention is estradiol valerate and/or drospirenone.

Further constituents of the pharmaceutical compositions may include conventional auxiliary substances such as for example antioxidants, binders, emulsifiers, colouring agents, filmforming agents, fillers, odoriferous substances, flavouring substances, gelforming agents, preservatives, solvents, oils, powder bases, ointment bases, acids and salts for the formulation, replenishment and production of pharmaceutical compositions, lubricants, release agents, suppository bases, suspension stabilisers, sweetening agents, effervescent gases, emollients and Bugar substitutes.

Plant medicament preparations and homeopathic preparations are also included among the pharmaceutical compositions in which the silicon dioxide Schülpen may be used.

The pharmaceutical compositions according to the invention may also include so-called retard and depot dosage forms with controlled release of active constituent. Moreover the pharmaceutical compositions according to the invention may also be part of therapeutic systems such as for example therapeutic systems for topical application and transdermal therapeutic systems.

In a preferred embodiment the silicon dioxide Schülpen based on pyrogenic silicic acid serves as a carrier for pharmaceutical active constituents and/or excipients. The present invention is accordingly also directed to an adsorbate of the aforedescribed silicon dioxide Schülpen and at least one of these substances.

The expression “adsorbate” as used in the present specification covers not only the adsorption of a substance on the surface of the silicon dioxide, but also in the pores, as well as the “incorporation” in the void volumes. The term “adsorbate” may also mean that silicon dioxide Schülpen or fragments thereof coat solid particles or liquid droplets of the material. In the latter case the forces of attraction between the particles and/or droplets are reduced and for example the flow behaviour is improved and/or the coalescence of droplets is prevented.

In principle the silicon dioxide Schülpen may act as a carrier for any suitable pharmaceutical active constituent or excipient; preferred however are adsorbates containing the aforementioned active constituents and auxiliary substances and/or their mixtures. Of the pharmaceutical excipients, there are preferably adsorbed on the silicon dioxide Schülpen fragrances, flavouring agents or colouring agents. The fragrances and flavouring agents may be of natural, i.e. plant or animal origin, as well as synthetic, i.e. fully synthetic or semi-synthetic origin.

Examples of plant fragrances include ethereal oils and resinoids. Examples of animal fragrances that may be mentioned include musk, civet, castoreum and ambergris. The fully synthetic fragrances include those that have an odoriferous prototype in nature, as well as pure fantasy compositions. Semisynthetic fragrances are understood to be those that can be isolated from natural sources and then chemically converted.

Also, the colouring agents may be natural or synthetic colouring agents, and organic or inorganic compounds.

Schülpen of pyrogenic silicic acid are suitable in particular as carriers for substances:
whose release behaviour is improved by application to a high surface area carrier substance, for example in the case of sparingly water-soluble substances;
whose release behaviour is too quick, for example in the case of retard formulations;
that are liquid or pasty and are therefore e.g. difficult to meter and/or handle;
that can be processed only with difficulty, for example as a result of too low a melting point;
whose flow behaviour is insufficient for further processing, for example for producing tablets and capsules;
that are readily volatile;
that are sensitive to external conditions such as for example atmospheric oxygen, light, moisture, acids (gastric juice) or bases (intestinal fluid).

Numerous active constituents can be stabilised in this way, such as for example acetylsalicylic acid; atropine; azulene; bisabolol; camphor; chloramphenicol; hydrocortisone and its derivatives, such as for example hydrocortisone-17-valerate, prostaglandins; thymol; (pro)vitamins and their derivatives, such as for example vitamin A and E; unsaturated fatty acids, specifically essential fatty acids such as for example gamma-linolenic acid, oleic acid, eicosapentenoic acid and docosahexenoic acid; extracts of animal and plant origin and oils of animal and plant origin, such as for example fish oils, evening primrose oil, borage oil, currant seed oil and cod liver oil.

Sparingly soluble substances whose release behaviour can be improved by application to the Schülpen formed from pyrogenic silicic acid include for example indomethacin, sulfaethidole, reserpine, griseofulvin, probucol and oxolinic acid. Also, the release behaviour of per se readily soluble substances such as for example hydrochlorothiazide, chloramphenicol and acetylsalicylic acid can be improved further in this way.

An example of an active constituent that is difficult to process or cannot be processed at all by conventional methods is ibuprofen, above all S-ibuprofen, which has a melting point of only 52° C. On account of the low melting point granulation processes apart from as an adsorbate according to the invention are hardly feasible. Moreover substances that for example sinter during the tabletting form preferred adsorbates within the context of the present invention with the silicon dioxide granular material.

The quantitative ratio of substance to silicon dioxide Schülpen in the adsorbate may be chosen as desired depending on the properties of the substance and the requirements that the end product has to meet. However, preferably 0.001 to 200 g of substance, particularly preferably 10 to 150 g of substance, are used per 100 g of silicon dioxide granular material.

Various procedures may be employed in order to apply and/or adsorb the desired active constituents and/or auxiliary substances on the silicon dioxide Schülpen. An exemplary process for the production of the adsorbate according to the invention comprises the following steps:
(a) melting of the substance(s) to be adsorbed, selected from pharmaceutical active constituents and auxiliary substances, or distribution, i.e. dissolution, suspension or emulsification, of the latter in a solvent;
(b) mixing the Schülpen based an pyrogenically produced silicon dioxide with the mixture from step (a); and
(c) optional removal of the solvent

The term “solvent” also includes mixtures of several different solvents. It is also understood that substances already liquid at room temperature can be subjected without prior processing to the mixing in step (b) since in this case the “melting process” has already taken place. The mixing step (b) may be carried out either by adding the mixture from step (a) to the silicon dioxide Schülpen, for example by spraying, or vice versa. In both cases the addition may take place in one amount or in portions. The duration of the mixing in step (b) depends above all on the adsorption behaviour of the substance to be adsorbed on the silicic acid surface. If a solvent is present, step (a) and step (b) are carried out at a temperature that is between the freezing point and boiling point of the solvent. The excess solvent that may be present is removed in step (c), preferably at elevated temperature and/or reduced pressure.

The removal of the solvent in step (c) may also be effected by spray drying or fluidised bed drying, a forming being carried out at the same time. In the case of a melt containing Schülpen the forming process may appropriately comprise an extrusion.

Schülpen formed from pyrogenic silicic acids may however also be used for the production of pharmaceutical preparations without their simultaneously acting as carriers and/or adsorption agents. In this case they can in particular complement or replace the conventional pyrogenic silicic acids that have been established in pharmaceutical practice for many years. For example, Schülpen of pyrogenic silicic acids may above all improve the production and properties of solid medicament forms. Also, they may advantageously be employed in the production of extrudates and replace for example other established auxiliary substances such as cellulose or polymers.

The advantages of the Schülpen based on pyrogenically produced silicon dioxide compared to the known non-granulated pyrogenic silicic acids lie above all in the higher bulk density and tamped density, improved flowability, narrower grain size distribution, and dust-free processing. In addition tablets produced therefrom have a higher mechanical stability and an improved disintegration behaviour.

The invention will now be described in more detail with the aid of examples.

Examples of Pharmaceutical Uses in Schülpen

Use as a Carrier for a Pharmaceutical Active Compound

For this, the AEROSIL Schülpen (silicon dioxide Schülpen) ideally should have no fine content. An AEROSIL Schülpen fraction (silicon dioxide Schülpen) having an AEROSIL Schülpen particle size (silicon dioxide Schülpen) of between 250 and 500 μm was prepared by a sieve granulation and subsequent sieving. This size range was chosen more or less arbitrarily. However, it proved to be particularly suitable in the use tests. In principle, however, it would also be possible to use smaller or larger particle sizes.

In this use example, the AEROSIL Schülpen (silicon dioxide Schülpen) and the comparison products were loaded with eucalyptus oil.

The following parameters were investigated to characterize the usability:
Flow properties of the loaded Schülpen (in glass cones, the diameter of the outflow opening was 2.5, 5, 8, 12 and 18 mm). The flow was evaluated with the ratings: very good=1, good=2, satisfactory=3, adequate=4, deficient=5 and inadequate=6.
Angle of the poured cone of the loaded Schülpen (this is calculated from the equation: tan(poured cone angle)=poured cone height/(0.5*poured cone diameter)
Capsule weight: For this, capsules were filled with loaded carrier material. It is important here that the highest possible weight can be metered into the capsules.
Deviation of the capsule weight: This is a measure of the metering accuracy.

The following were used for comparison:

AEROSIL 300 V, which is a highly disperse pulverulent pyrogenically produced silicon dioxide.

AEROPERL Pharma 30, which is a spray-dried pyrogenically produced silicon dioxide.

TABLE
Flow properties of the AEROSIL Schülpen compared with the starting
substance of the compacting (AEROSIL 300 V) and VP AEROPERL 300 Pharma 30

| | Content of | Content | | Angle of the | Bulk | Capsule | Deviation of the |
|---|---|---|---|---|---|---|---|
| | Silica | of | Flow | poured cone | density | weight | capsule weight |
| Product | [%] | oil [%] | properties | [°] | [g/ml] | [mg] | [%] |
| AEROSIL 300 V | 50 | 50 | 5-6 | 47 | 0.26 | 191 | 2.50 |
| (starting substance | 43* | 55 | 6 | 49 | 0.33 | 230 | 2.24 |
| for the granulation) | 40 | 60 | 5-6 | 50 | 0.44 | 245 | 2.66 |
| AEROSIL Schülpen | 50 | 50 | 1 | 39 | 0.54 | 351 | 0.97 |
| (250 μm < x < 500 μm) | 45 | 55 | 1 | 29 | 0.60 | 366 | 1.07 |
| VP AEROPERL 300 | 50 | 50 | 2 | 36 | 0.46 | 292 | 1.33 |
| Pharma 30 | 40 | 60 | 2 | 34 | 0.57 | 350 | 0.93 |

*45 parts oil resulted in a cohesive mass that could not be tested
**- cohesive mass, could not be tested

The AEROSIL Schülpen are fascinating above all due to outstanding flow properties compared with the starting material AEROSIL 300 V. This is to be explained by the fact that the Schülpen particles do not agglomerate like the aggregates of the highly disperse pyrogenic silica AEROSIL 300 V, and take up and store the model active compound (eucalyptus oil) in their pore volume. As a result, they do not stick and remain very readily free-flowing.

The AEROSIL Schülpen also have slightly improved flow properties compared with AEROPERL. Compared with AEROPERL, the AEROSIL Schülpen have the advantage of a lack of dust, see FIG. 7.

A disadvantage of working with highly disperse silicon dioxide is, in particular, the dust formation, since the highest purity requirements must be met in the manufacture of pharmaceutical and cosmetic products.

1. A pharmaceutical composition comprising a pharmaceutically active constituent and as an auxilliary substance Schülpen of pyrogenically produced silicon dioxide.

2. The pharmaceutical composition according to claim 1, wherein the pharmaceutical composition is present in the form of a suspension, emulsion, aerosol, ointment, cream, gel, paste, suppository, stick, powder, topical powder, redispersible powder, powder used to make oral suspsensions, granules, tablet, pastille, sugar-coated pill, film-coated tablet, filled hard gelatin capsule, soft gelatin capsule, extrudate, microcapsule or a microsphere.

3. The pharmaceutical composition according to claim 1, wherein the Schülpen act as a carrier for pharmaceutical active constituents and/or excipients.

4. Pharmaceutical composition containing Schülpen based on pyrogenically produced silicon dioxide and at least one pharmaceutical active constituent.

5. Pharmaceutical composition according to claim 4, wherein it furthermore contains at least one pharmaceutical excipient.

6. Pharmaceutical composition according to claim 5, wherein the pharmaceutical excipient is selected from the group consisting of: antioxidants, binders, emulsifiers, colouring agents, filmforming agents, fillers, gel-forming agents, odoriferous substances, flavouring substances, preservatives, solvents, oils, powder bases, ointment bases, acids and salts for the formulation, replenishment and production of pharmaceutical compositions, lubricants, release agents, suppository bases, suspension stabilisers, sweetening agents, effervescent gases, emollients and sugar substitutes.

7. Adsorbate of Schülpen based on pyrogenically produced silicon dioxide and at least one further substance selected from the group consisting of pharmaceutical active constituents and excipients.

8. Adsorbate according to claim 7, wherein the pharmaceutical excipient is selected from the group consisting of: antioxidants, binders, emulsifiers, colouring agents, filmforming agents, fillers, gel-forming agents, odoriferous substances, flavouring substances, preservatives, solvents, oils, powder bases, ointment bases, acids and salts for the formulation, replenishment and production of pharmaceutical compositions, lubricants, release agents, suppository bases, suspension stabilisers, sweetening agents, effervescent gases, emollients and sugar substitutes.

9. Process for the production of an adsorbate according to claim 7, comprising the following steps:
(a) melting the substance(s) to be adsorbed, selected from pharmaceutical active constituents and excipients or distribution thereof in the solvent;
(b) mixing the granular material based an pyrogenically produced silicon dioxide with the mixture from step (a); and
(c) optionally removal of the solvent.
